(19) United States
(12) Patent Application Publication
(10) Pub. No.: US 2009/0125117 A1
(43) Pub. Date: May 14, 2009

(54) LEG ALIGNMENT AND LENGTH MEASUREMENT IN HIP REPLACEMENT SURGERY

(76) Inventors: Francois Paradis, Boucherville (CA); Bruno Falardeau, Verdun (CA)

Correspondence Address: OGILVY RENAULT LLP, 1981 MCGILL COLLEGE AVENUE, SUITE 1600, MONTREAL, QC, H3A2Y3, CA

(21) Appl. No.: 12/271,246

(22) Filed: Nov. 14, 2008

Related U.S. Application Data
(60) Provisional application No. 60/987,888, filed on Nov. 14, 2007.

(51) Int. Cl. A61F 2/32; A61B 17/56; A61B 5/05

(52) U.S. Cl. 623/22.11; 606/102; 600/426

(57) ABSTRACT

A CAS system for measuring surgical parameters during hip replacement surgery comprises a first tracking reference in fixed relation with the pelvis to form a frame of reference. A registration tool is trackable. A sensor apparatus tracks the first tracking reference and the registration tool. A controller unit receives tracking data for the first trackable reference and the registration tool. The controller unit has a position and orientation calculator for calculating from the tracking data a position and orientation of the pelvic tracking reference to track the frame of reference, and of the registration tool to produce femoral models at a first and a second sequential operative steps. An alignment adjustor aligns the femoral models with the frame of reference. A surgical parameter calculator calculates surgical parameters as a function of the femoral models as aligned by the alignment adjustor.

### CROSS-REFERENCE TO RELATED APPLICATION

This patent application claims priority on U.S. Provisional Patent Application No. 60/987,888, filed on Nov. 14, 2007.

### FIELD OF THE APPLICATION

The present application generally relates to computer-assisted hip replacement surgery and, more precisely, to surgical parameter measurement and adjustment in hip replacement surgery.

### BACKGROUND OF THE ART

Total hip replacement surgery involves the introduction of an artificial hip joint in a patient. The artificial hip joint typically consists of a pelvic implant and a femoral implant. The pelvic implant is a cup received in the acetabulum. The femoral implant consists of a spherical portion received at an end of a longitudinal implant portion, or a femoral implant secured to the resurfaced femoral head. In the first case, the longitudinal implant portion is introduced into the intramedullary canal of the resected femur, with the spherical portion being generally centered with respect to the previous position of the femoral head. Therefore, the femoral head (i.e., spherical portion of the femoral implant) and the cup (i.e., pelvic implant) coact to create the artificial hip joint.

Different output values are of concern in hip replacement surgery. In order to reproduce a natural and/or improved gait and range of motion to a patient, the position and orientation of the implants, the medio-lateral offset of the femur and the limb length discrepancy must be considered during surgery. The work of the surgeon during hip replacement surgery will have a direct effect on these output values, and a successful surgery will relieve pain, provide motion with stability and correct deformities.

There is no precise definition of the intra-implanting limb length discrepancy (hereinafter “intraop-LLD”) and intra-implanting medio-lateral offset (hereinafter “intraop-MLO”). On the preoperative X-rays, surgeons usually measure preoperative limb length discrepancy (hereinafter “preop-LLD”) along the vertical axis of the body as a relation between the interischial line of the pelvis and the lesser trochanter of the femur. During the intra-implanting period, in order to obtain reasonable measurements that are then possible to validate with X-ray measurements, the surgeons have to align the leg along the vertical axis of the body. This alignment is highly dependent on the surgeon skills and experience. Changes in adduction/abduction of the leg will significantly alter the measurement and introduce measurements errors.

The accuracy of the measurements rests heavily on the surgeon's ability to reposition the leg accurately before each measurement. Therefore, in order to obtain an accurate intraop-LLD and intraop-MLO measurement, the leg, after the implant reduction, must be realigned in the same orientation as before the dislocation. Again, changes in adduction/abduction, flexion/extension and rotation of the leg will significantly alter the measurement.

Failure to provide a robust and accurate method for leg length and offset measurement intraoperatively might lead to the postoperative leg length inequality. This in turn might lead to patient dissatisfaction and/or discomfort, functional impairment (low back pain, static nerve palsy, abductor weakness, dysfunctional gait), unstable hip joint, early mechanical loosening.

### SUMMARY OF THE APPLICATION

It is an aim of the present application to address the issues pertaining to the prior art.

It is a further aim of the present invention to provide a novel method for guiding an operator in measuring surgical parameters such as limb length discrepancy and medio-lateral offset intraoperatively in computer-assisted surgery.

Therefore, in accordance with a first embodiment, there is provided a method of measuring surgical parameters in computer-assisted surgery so as to guide an operator in inserting a hip joint implant into a femur, comprising: creating a frame of reference for the femur as a function of a pelvic tracking reference; digitizing a first femoral model with respect to the frame of reference; digitizing a second femoral model with respect to the frame of reference after initiation of hip joint replacement; aligning the second femoral model and the first femoral model with the frame of reference; and calculating surgical parameters as a function of the alignment of the femoral models in the frame of reference.

Further in accordance with the first embodiment, creating the frame of reference comprises obtaining a normal of a plane supporting the patient in a given posture for the frame of reference of the femur.

Still further in accordance with the first embodiment, creating the frame of reference comprises positioning the patient in lateral decubitus, and registering the normal of the plane as a medio-lateral axis of the patient.

Still further in accordance with the first embodiment, creating the frame of reference comprises digitizing a mechanical axis and projecting the mechanical axis on the plane to define a longitudinal axis, with the longitudinal axis and the normal of the plane forming two axes of the frame of reference, a frontal plane of the frame of reference being defined as parallel to the two axes.

Still further in accordance with the first embodiment, digitizing the first femoral model comprises digitizing an anatomical axis for the femur, the anatomical axis passing through a point on the greater trochanter and any one of a point on the patella and a midpoint of the condyles.

Still further in accordance with the first embodiment, digitizing the first femoral model comprises digitizing a mechanical axis for the femur, the mechanical axis passing through a center of rotation of the femur and any one of a point on the patella and a midpoint of the condyles.

Still further in accordance with the first embodiment, creating the frame of reference comprises using the mechanical axis of the first femoral model to create the frame of reference.

Still further in accordance with the first embodiment, digitizing the second femoral model comprises digitizing said anatomical axis for the femur after initiation of hip joint replacement with the same points as the first femoral model.

Still further in accordance with the first embodiment, digitizing the second femoral model comprises digitizing the mechanical axis for the femur after initiation of hip joint replacement with the same points as the first femoral model, with a center of rotation being as a function of at least one of a pelvic implant and a femoral implant.

Still further in accordance with the first embodiment, aligning the first femoral model with the frame of reference comprises (1) rotating the first femoral model with respect to a center of rotation taken before initiation of hip joint replacement such that a mechanical axis of the first femoral model is parallel to a longitudinal axis of the frame of reference, and (2) rotating a femoral plane of the first femoral model with respect to the mechanical axis of the first femoral model such that the femoral plane of the first femoral model is parallel to a frontal plane of the frame of reference; and aligning the second femoral model with the frame of reference comprises (1) rotating the second femoral model with respect to a center of rotation taken after initiation of hip joint replacement such that a mechanical axis of the second femoral model is parallel to the longitudinal axis of the frame of reference, and (2) rotating a femoral plane of the second femoral model with respect to the mechanical axis of the second femoral model such that the femoral plane of the second femoral model is parallel to the frontal plane of the frame of reference.

Still further in accordance with the first embodiment, digitizing the first femoral model comprises digitizing the mechanical axis, an anatomical axis, and the femoral plane in which both the mechanical axis and the anatomical axis lie.

Still further in accordance with the first embodiment, digitizing the second femoral model comprises digitizing the mechanical axis, an anatomical axis, and the femoral plane in which both the mechanical axis and the anatomical axis lie, after initiation of hip joint replacement.

Still further in accordance with the first embodiment, calculating surgical parameters comprises calculating a limb length discrepancy by projecting a mechanical axis of the first femoral model on a plane supporting the patient to form a longitudinal axis, and by measuring a distance on the longitudinal axis between a femoral landmark point taken before and after initiation of hip joint replacement.

Still further in accordance with the first embodiment, calculating surgical parameters comprises calculating a medio-lateral offset by measuring a distance on the medio-lateral axis between a femoral landmark point taken before and after initiation of hip joint replacement.

In accordance with a second embodiment, there is provided a CAS system for measuring surgical parameters during hip replacement surgery to guide an operator in inserting a hip joint implant into a femur, comprising: at least a first tracking reference in fixed relation with the pelvis, the first tracking reference being trackable to form a frame of reference; a registration tool being trackable; a sensor apparatus for tracking at least the first tracking reference and the registration tool; and a controller unit for receiving tracking data for at least the first trackable reference and the registration tool, the controller unit having: a position and orientation calculator for calculating from the tracking data a position and orientation of at least the pelvic tracking reference to track the frame of reference, and of the registration tool to produce femoral models at a first and a second sequential operative steps; an alignment adjustor for receiving the femoral models as a function of at least the first tracking reference, and for aligning the femoral model of the second operative step and the femoral model of the first operative step with the frame of reference; and a surgical parameter calculator for calculating surgical parameters as a function of the femoral model of the first operative step and the femoral model of the second operative step as aligned by the alignment adjustor.

Further in accordance with the second embodiment, the position and orientation calculator produces the frame of reference from a normal of a plane supporting the patient in a given posture.

Still further in accordance with the second embodiment, the frame of reference comprises a medio-lateral axis being the normal of the plane when the patient is in lateral decubitus.

Still further in accordance with the second embodiment, the position and orientation calculator produces the frame of reference from a mechanical axis of the femur, with a projection of the mechanical axis on the plane and the normal of the plane forming two axes of the frame of reference.

Still further in accordance with the second embodiment, the first femoral model comprises an anatomical axis for the femur passing through a point on the greater trochanter and any one of a point on the patella and a midpoint of the condyles.

Still further in accordance with the second embodiment, the first femoral model comprises a mechanical axis passing through a center of rotation of the femur and any one of a point on the patella and a midpoint of the condyles.

Still further in accordance with the second embodiment, the frame of reference comprises the mechanical axis of the first femoral model.

Still further in accordance with the second embodiment, the second femoral model comprises said anatomical axis for the femur as obtained after initiation of hip joint replacement with the same points as the first femoral model.

Still further in accordance with the second embodiment, the second femoral model comprises the mechanical axis for the femur as obtained after initiation of hip joint replacement with the same points as the first femoral model, with a center of rotation being as a function of at least one of a pelvic implant and a femoral implant.

Still further in accordance with the second embodiment, the alignment adjustor aligns the first femoral model with the frame of reference by (1) rotating the first femoral model with respect to a center of rotation taken before initiation of hip joint replacement such that a mechanical axis of the first femoral model is parallel to a longitudinal axis of the frame of reference, and by (2) rotating a femoral plane of the first femoral model with respect to the mechanical axis of the first femoral model such that the femoral plane of the first femoral model is parallel to a frontal plane of the frame of reference; and the alignment adjustor aligns the second femoral model with the frame of reference by (1) rotating the second femoral model with respect to a center of rotation taken after initiation of hip joint replacement such that a mechanical axis of the second femoral model is parallel to the longitudinal axis of the frame of reference, and by (2) rotating a femoral plane of the second femoral model with respect to the mechanical axis of the second femoral model such that the femoral plane of the second femoral model is parallel to the frontal plane of the frame of reference.

Still further in accordance with the second embodiment, the first femoral model comprises the mechanical axis, an anatomical axis, and the femoral plane in which both the mechanical axis and the anatomical axis lie.

Still further in accordance with the second embodiment, the second femoral model comprises the mechanical axis, an anatomical axis, and the femoral plane in which both the mechanical axis and the anatomical axis lie, as obtained after initiation of hip joint replacement.

Still further in accordance with the second embodiment, the surgical parameter calculator calculates a limb length discrepancy by projecting a mechanical axis of the first femoral model on a plane supporting the patient to form a longitudinal axis, and by measuring a distance on the longitudinal axis between a femoral landmark point taken before and after initiation of hip joint replacement.

Still further in accordance with the second embodiment, the surgical parameter calculator calculates a medio-lateral offset by measuring a distance on the medio-lateral axis between a femoral landmark point taken before and after initiation of hip joint replacement.

### BRIEF DESCRIPTION OF THE DRAWINGS

FIG. 1 is a front elevation view of bones involved in a hip replacement method in accordance with the present application;

FIG. 2 is a flow chart of a method of hip replacement surgery in accordance with an embodiment of the present application; and

FIG. 3 is a block diagram of a computer-assisted surgery system performing a hip replacement surgery in accordance with another embodiment of the present application.

### DESCRIPTION OF THE PREFERRED EMBODIMENTS

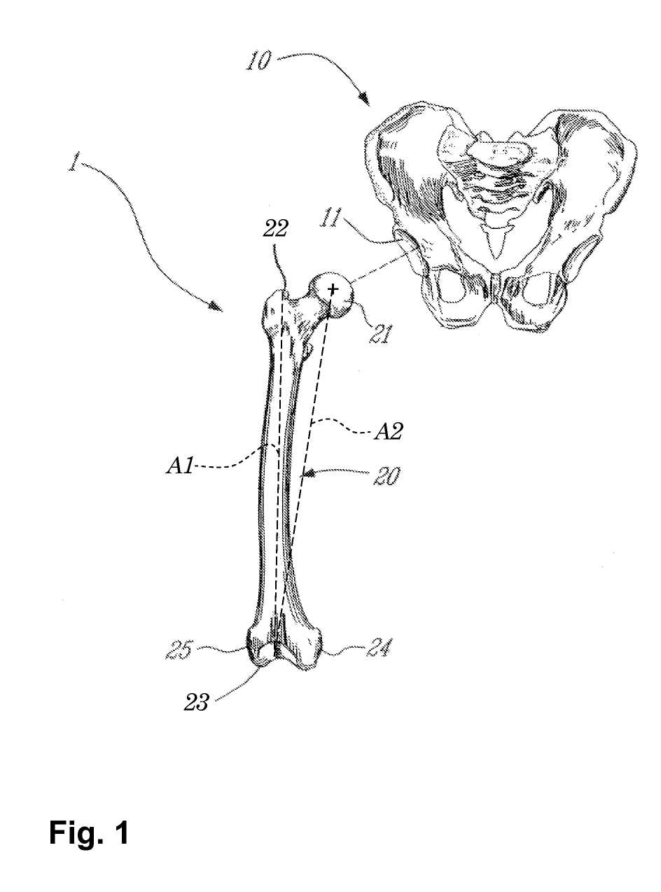

According to the drawings, and more particularly to FIG. 1, bones of the leg that are involved in the hip replacement surgery are generally shown at 1. FIG. 1 is provided as reference for the description of the steps of surgical parameter measurements associated with the hip replacement surgery method described herein. The bones are the pelvis 10, and the femur 20. One of the acetabulums of the pelvis 10 is illustrated at 11. The femur 20 has a femoral head 21, a greater trochanter featuring landmark 22. The patella (not shown is in region 23 of the femur 20. The epicondyles 24 and 25 are on opposite sides of the femur 20.

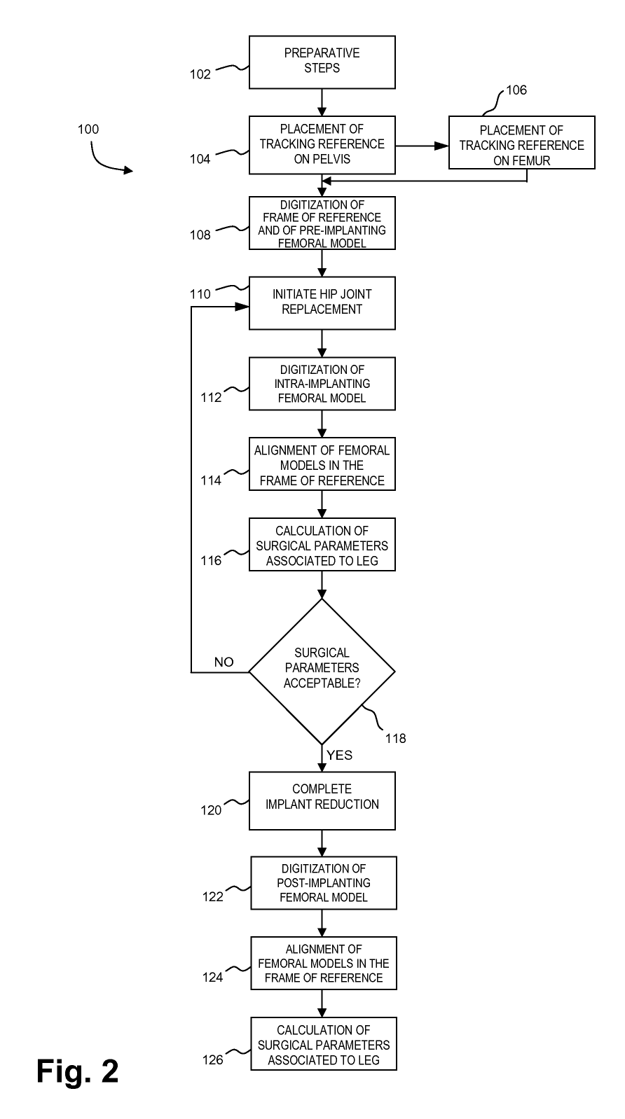

Referring to FIG. 2, a method for hip replacement surgery incorporating surgical parameter measurements in accordance with the present application is generally shown at 100. It is pointed out that the method 100 is a hip replacement method. Accordingly, the method 100 is associated with existing hip replacement methods, such as the method described in United States Publication No. 2004/0230199 by Jansen et al., published Nov. 18, 2004. Moreover, although the method 100 is described with a given sequence of steps, some digitizing steps may be suitably switched with surgical steps in accordance with the surgical method chosen by the operator. A plurality of methods can be derived from the method 100 according to the decisions of the surgeon. For instance, United States Patent Application Publication No. 2006/0293614, by the present assignee, describes a surgical method involving leg alignment in hip replacement surgery, for surgical parameter measurement. This patent application is hereby incorporated by reference.

It is pointed out that the following definitions will be used in this document: pre-implanting refers to the period before the resection of the bones of the hip joint, intra-implanting refers to the period after resection and before reduction, and post-implanting refers to the period after the final implants have been fixed to the bones.

In Step 102, preparative steps for surgery are effected. Namely, general patient information can be entered into the CAS system for opening a patient file. For instance, a general patient profile can be entered, that can consist of the name, birth date, identification number, sex and the like, as well as more specific data pertaining to the surgery, such as preoperative leg length discrepancy (with the identification of the longer leg), if applicable. For instance, the preoperative leg length discrepancy is measured using X-rays of the hip joint. More precisely, the leg length discrepancy is measured from the vertical comparison between the trochanters. These X-rays are typically taken during the diagnostic stages leading to surgery, so they are usually available for hip joint surgery. The calibration of the various surgical tools to be used is done. For instance, a calibration base and method, as set forth in International Publication No. WO 01/67979 A1 by Jutras et al., can be used for the calibration. Also, correspondence between the tracking of the tools and the display on the CAS controller (both described hereinafter) can be verified in further calibration steps included in Step 102.

It is pointed out that the general patient information can be entered preoperatively. Moreover, the entering of the general patient information is straightforward such that the surgeon need not be involved. However, in order to minimize the preoperative procedures, all preparative steps of method can be performed at the beginning of the surgical session, during a short time span preceding the surgery.

In Step 104, a tracking reference is secured to the pelvis 10 and is referred to hereinafter as the pelvic tracking reference. Therefore, the pelvis 10 can be tracked for position and orientation in space as a function of the tracking reference, by the CAS system. The tracking reference will remain anchored to its respective bone (if applicable) throughout the computer-assisted steps of surgery.

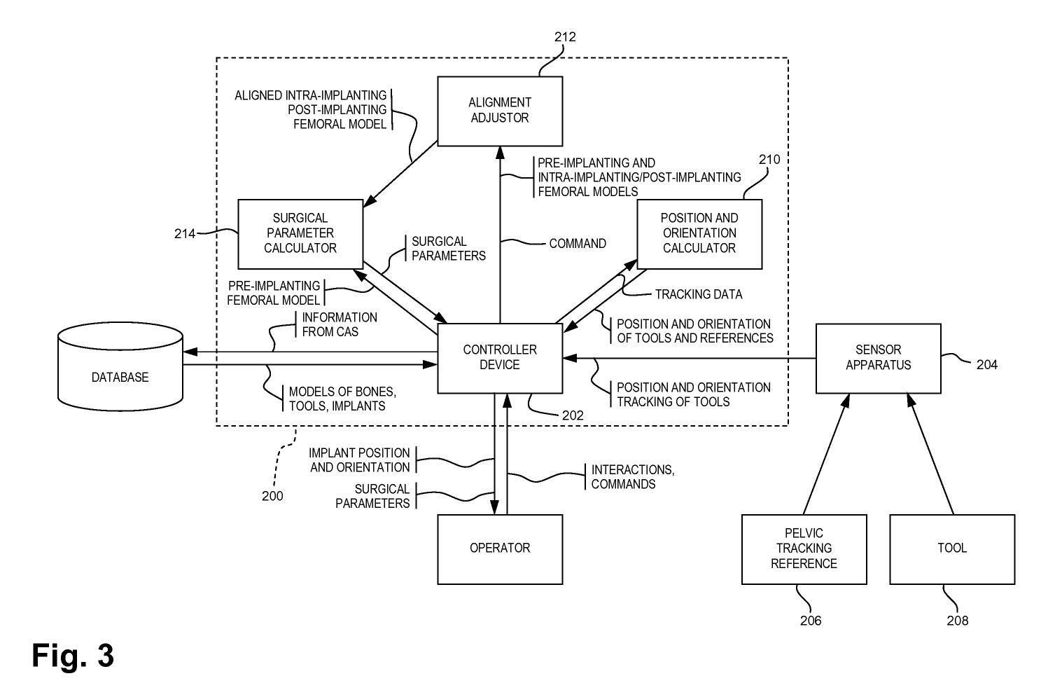

In Step 106, another tracking reference is secured to the femur 20, for the tracking thereof for position and orientation. In order to reduce the invasiveness of the surgery, the use of a femoral tracking reference is optional, hence Step 106 is optional, as is illustrated in FIG. 3. Accordingly, the method 100 will be described as not involving any femoral reference.

Surgery is initiated between Step 104 and subsequent Step 106 or 108, by the surgeon exposing the hip joint while the patient is positioned in a given posture, such as lateral decubitus. No computer assistance is required thereat.

Step 108 consists in the digitization of a frame of reference and of a pre-implanting femoral model. To effect Step 108 and subsequent steps, the patient preferably lies in a lateral decubitus position on the operating room table (i.e., OR table), although other positions (e.g., patient lying on his/her back) can be used as well. As the lateral decubitus position is preferred, the following sequence of steps will be described for the patient in the lateral decubitus position.

As a first substep in digitizing the pre-implanting frame of reference, the OR table plane is digitized, so as to define an OR table frame of reference with respect to the pelvic tracking reference.

The OR table is digitized with a registration pointer from the tools. Various methods can be used to define the OR table plane. In one contemplated embodiment, three points are taken on the table or on a surface parallel to the plane of the OR table. The points that are acquired must form a triangular geometry (i.e., they cannot be linear) so as to define the plane of the OR table. Considering that the body is at that moment in lateral decubitus, the normal of the table plane will then be considered as the medio-lateral axis of the body.

Other contemplated embodiments include the use of a tracked plate that can be laid down on the OR table or a surface parallel to the OR table to find its normal, to then define the medio-lateral axis of the body. Therefore, by registering the normal of the table plane as medio-lateral axis, it is assumed that there is a negligible offset of the body in the lateral decubitus position. The medio-lateral axis is part of the frame of reference.

Still in Step 108, a registration of the anatomical axis A1 of the femur 20 is performed. In order to register the anatomical axis, the femur is physically aligned with the longitudinal axis of the body before the registration occurs. The anatomical axis A1 is determined by the registration of points on the greater trochanter 22 and on the patella, which points are preferably marked for subsequent use. As an alternative to the patella, a midpoint between the medial and lateral epicondyles 24 and 25 can be calculated.

Still in Step 108, a registration of the mechanical axis A2 of the femur 20 is performed with respect to the pelvic tracking reference. The mechanical axis A2 features the center of rotation of the hip joint. The digitization of the center of rotation of the hip joint will be dependent on the number of tracking references, as exposed above (either one or two tracking references).

If only one tracking reference is used, namely on the pelvis, a temporary tracking reference is positioned in a stable manner to the femur 20, and rotational movements of the femur 20 with respect to the pelvis 10 are performed. Accordingly, these movements will enable the CAS system 50 to calculate a center of rotation of the hip joint 10, and an assumption is then made that the center of rotation of the femur 20 is coincident with the center of rotation of the acetabulum 11. The calculated center of rotation of the hip joint will then be associated with the tracking reference on the pelvis. It is also contemplated to track a reamer from the tools so as to obtain, in view of the geometry of the reamer, a position for the center of rotation of the acetabulum 11.

Another method contemplated for obtaining the center of rotation of the hip joint 10 is to digitize points in the acetabulum 11 with respect to the tracking reference on the pelvis 10, which requires that the femur is dislocated or resected to expose the acetabulum 11. This method also assumes that the centers of rotation of the femur and the pelvis are coincident. Some references, such as U.S. Publication No. 2004/0230199, have already exposed this method of obtaining the center of rotation of the hip joint 10.

The axis going through the center of rotation and the digitized landmark on the patella (i.e., obtained during the digitization of the anatomical axis A1) forms the mechanical axis of the femur 20. A plane in which both the anatomical axis A1 and the mechanical axis A2 lie is registered with respect to the pelvic tracking reference, and is referred to as the pre-implanting femoral plane. The pre-implanting femoral plane, the anatomical axis A1 and the mechanical axis A2 define the pre-implanting femoral model.

The projection of the mechanical axis A2 on the table plane defines the longitudinal axis along which will be measured the leg length discrepancies. The medio-lateral axis of the femur (i.e., the normal of the table), the longitudinal axis and the cross product of these axes (i.e., the reference frontal plane) form the frame of reference related to the pelvic tracking reference. Moreover, a frontal plane of the patient is the plane in which both the longitudinal axis and the medio-lateral axis lie. Parameters such as the leg length discrepancy and the offset will be measured during the intra-implanting period based on the pre-implanting femoral model.

Other surgical parameter measurements will be based upon the pre-implanting femoral model. For instance, the point on the greater trochanter, as obtained when defining the anatomical axis A1 of the femur 20 in Step 108, can be used as a landmark for the calculation of medio-lateral offset and limb length discrepancy from pre-implanting, intra-implanting, as well as post-implanting data.

In Step 110, the replacement of the hip joint is initiated. As mentioned previously, the Step 110 is dependent on the method of surgery chosen by the operator. Accordingly, few details are given herein, but reference is made to U.S. Publication No. 2004/0230199, in which a suitable method for performing the implant reduction is described.

After implant reduction, the operator will need surgical parameter measurements to validate the work being performed. The alterations to the femur 20 and the acetabulum 11 will result in potential changes to the position of the center of rotation of the hip joint 10.

It is therefore necessary to digitize an intra-implanting femoral model. This involves redigitizing the center of rotation to perform the surgical parameter measurements with respect to the landmark points (e.g., the anatomical axis point on the greater trochanter).

Therefore, Step 112 involves the digitization of an intra-implanting center of rotation for the hip joint 10 so as to redefine the mechanical axis (i.e., the intra-implanting mechanical axis).

For instance, alterations may be performed to the acetabulum 11 in addition to the insertion of a femoral implant to replace the femoral head 21. In both these cases, the implants will potentially change the position of the center of rotation of the acetabulum 11 and the femoral head 21.

Therefore, in order to redigitize the center of rotation of the acetabulum 11 if an acetabular implant is used, a calibration tool can be inserted into the implanted hip joint so as to obtain the center of rotation of the acetabulum 11. One such calibration tool is described in International Publication No. WO 2005/023110, by the present assignee.

For the center of rotation of the femur 20, physical models of femoral implant are often provided to the operator for the modelization of the center of rotation of the femur 20. More specifically, the physical models represent different sizes of femoral implant, and are used to temporarily estimate the leg length and medio-lateral offset.

With such physical models, the femur 20 is readily digitized, for instance, by digitizing surface points on the physical model inserted into the femur 20, or by reproducing a motion of the femur 20 with respect to the pelvis, with a tracking reference secured or positioned on the femur 20.

It is therefore required to have a center of rotation for the acetabulum 11, assumed to be the hip joint and possibly acquired with the calibration tool while the joint is dislocated, which center of rotation will be used subsequently in the alignment of the femur 20 in a selected orientation.

The leg is reduced with its implant, and at least two points on the femur 20, excluding the femoral implant (i.e. ball head) center, must be digitized in Step 112 so as to complete the intra-implanting femoral coordinate system. It is contemplated to mark points (using a cortical screw, a sterile pen, an electro-cutter, amongst other possibilities) on the bone during the digitization of the pre-implanting femoral coordinate system in Step 108. A registration pointer will be used to digitize these known points. It is pointed out that it is important to have the femur 20 immobilized when taking these points. Once these points are confirmed, the intra-implanting anatomical axis and mechanical axis are known with respect to the pelvic tracking reference. The intra-implanting mechanical axis passes through the new center of rotation and the landmark on the patella, whereas the intra-implanting anatomical axis passes through the landmarks on the greater trochanter and on the patella, and thus in similar fashion to the axes A1 and A2. Moreover, an intra-implanting femoral plane is defined as the plane in which both the intra-implanting mechanical and anatomical axes lie, whereby the intra-implanting femoral model is completed.

In Step 114, the pre-implanting and intra-implanting femoral models are aligned with the frame of reference defined in Step 108, for comparative measurements to be calculated. In a first substep of alignment, the pre-implanting mechanical axis is rotated about the pre-implanting center of rotation until it is parallel to the longitudinal axis of the frame of reference. In other words, the pre-implanting mechanical axis is rotated in the frontal plane (Step 108) until it is parallel to the longitudinal axis. In a second substep of alignment, the pre-implanting femoral plane is rotated about the pre-implanting mechanical axis until it is parallel to the frontal plane of the frame of reference obtained in Step 108. These two steps are then repeated for the intra-implanting femoral model.

Once the alignment is completed, surgical parameters are calculated in Step 116, using the pre-implanting femoral model and the aligned intra-implanting femoral model with respect to the frame of reference. The limb length discrepancy can be calculated on the longitudinal axis performed in Step 108 as the spacing between the pre-implanting and the intra-implanting or post-implanting landmark (e.g., greater trochanter). Similarly, the medio-lateral offset can be calculated as the difference between the position of the landmarks along the medio-lateral axis obtained in Step 108.

Accordingly, information will be provided to the operator, so as to guide the operator in the alterations to be performed on the femur 20 in view of the calculated surgical parameters.

In Decision 118, the limb length discrepancy and the medio-lateral offset calculated in Step 116 may prompt adjustment in Step 110 of replacement of the hip joint. Ultimately, acceptable limb length discrepancy and medio-lateral offset will lead to Step 120 with the completion of the replacement of the joint.

Steps 122, 124 and 126 relate to the calculation of post-implanting surgical parameters. Following the description of Steps 112, 114 and 116 respectively, Steps 122, 124 and 126 are performed to obtain limb length discrepancy and medio-lateral offset from final measurements taken on the implants.

Various parameters considered during the method 100 are described below. The target leg length is a desired position for the femoral center of rotation, and is calculated as follows:

(target leg length)=Δ_{LL x-ray}+adjustment value

where (Δ_{LL x-ray}) is the initially acquired limb length discrepancy from the preoperative X-rays as described previously. The adjustment value is any value selected by the operator to correct the target leg length in view of the initially acquired limb length discrepancy.

Another guiding parameter to be provided to the surgeon is the current leg length discrepancy. The current leg length discrepancy, (current Δ_{LL}), is calculated as follows:

(current Δ_{LL})=(GTᵢₙₜᵣₐₒₚ)−(GTₚᵣₑₒₚ)−(target leg length),

where (GTᵢₙₜᵣₐₒₚ) is the intra-implanting Z value (Z value is the given by the longitudinal component of a position) of the greater trochanter point following the alignment procedure, (GTₚᵣₑₒₚ) is the pre-implanting Z value of the greater trochanter point following the realignment procedure, and where (target leg length) has been calculated previously. The current leg length discrepancy can be displayed by the CAS system 50 as an overall leg length, or as a relative value between leg lengths, with the value 0 representing legs of equal length.

Another guiding parameter to be provided to the surgeon is the current medio-lateral offset. The current medio-lateral offset, (current ΔMLO), is calculated as follows:

(current ΔMLO)=(GTᵢₙₜᵣₐₒₚ)−(GTₚᵣₑₒₚ)

where (GTᵢₙₜᵣₐₒₚ) is the intra-implanting X value (the X value is the given by the media-lateral component of a position) of the greater trochanter point following the realignment procedure (Step 116), (GTₚᵣₑₒₚ) is the pre-implanting X value of the greater trochanter point following the realignment procedure.

Now that the method 100 has been described in detail, the CAS system 200 will be described in accordance with the preferred embodiment of the present invention.

Referring to FIG. 4, the CAS system 200 is shown in greater detail. The CAS system 200 typically is a processing unit having a controller device 202 which processes the information. The controller device 202 is connected to a sensor apparatus 204 so as to receive position and orientation tracking data of tools, such as the pelvic tracking reference 206 (Step 104) and tools 208 such as a registration tool.

A position and orientation calculator 210 receives the tracking data, and calculates position and orientation of tools, as well as femoral models (e.g., the pre-implanting and intra-implanting femoral models) as a function of the pelvic tracking reference 206. Therefore, the controller device 202 allows the operator to perform the surgery in real-time CAS navigation.

In order to compare points taken during the pre-implanting period with points taken in the intra-implanting and post-implanting periods according to Steps 114 and 124 of the method 100, a alignment adjustor 212 is provided in association with the controller device 200. More specifically, data associated with the pre-implanting and intra-implanting/post-implanting femoral models is received by the alignment adjustor 212. When the information is complete (i.e., both pre-implanting and intra/post-implanting femoral models have been created), the alignment adjustor 212 aligns the intra/post-implanting models according to Step 114/124. As mentioned previously, the alignment adjustment consists in positioning the intra/post-implanting mechanical axis (with post-implanting center of rotation) parallel to the pre-implanting mechanical axis and then rotating the intra/post-implanting femoral plane until it is parallel with the pre-implanting femoral plane.

The aligned intra/post-implanting femoral model is then provided to a surgical parameter calculator 214, which will calculate surgical parameters comparing the femoral models as described for steps 116 and 126. Therefore, no physical alignment is required considering that the CAS system 200 performs all alignment virtually.

1. A method of measuring surgical parameters in computer-assisted surgery so as to guide an operator in inserting a hip joint implant into a femur, comprising:
creating a frame of reference for the femur as a function of a pelvic tracking reference;
digitizing a first femoral model with respect to the frame of reference;
digitizing a second femoral model with respect to the frame of reference after initiation of hip joint replacement;
aligning the second femoral model and the first femoral model with the frame of reference; and
calculating surgical parameters as a function of the alignment of the femoral models in the frame of reference.

2. The method according to claim 1, wherein creating the frame of reference comprises obtaining a normal of a plane supporting the patient in a given posture for the frame of reference of the femur.

3. The method according to claim 2, wherein creating the frame of reference comprises positioning the patient in lateral decubitus, and registering the normal of the plane as a medio-lateral axis of the patient.

4. The method according to claim 3, wherein creating the frame of reference comprises digitizing a mechanical axis and projecting the mechanical axis on the plane to define a longitudinal axis, with the longitudinal axis and the normal of the plane forming two axes of the frame of reference, a frontal plane of the frame of reference being defined as parallel to the two axes.

5. The method according to claim 1, wherein digitizing the first femoral model comprises digitizing an anatomical axis for the femur, the anatomical axis passing through a point on the greater trochanter and any one of a point on the patella and a midpoint of the condyles.

6. The method according to claim 1, wherein digitizing the first femoral model comprises digitizing a mechanical axis for the femur, the mechanical axis passing through a center of rotation of the femur and any one of a point on the patella and a midpoint of the condyles.

7. The method according to claim 6, wherein creating the frame of reference comprises using the mechanical axis of the first femoral model to create the frame of reference.

8. The method according to claim 5, wherein digitizing the second femoral model comprises digitizing said anatomical axis for the femur after initiation of hip joint replacement with the same points as the first femoral model.

9. The method according to claim 6, wherein digitizing the second femoral model comprises digitizing the mechanical axis for the femur after initiation of hip joint replacement with the same points as the first femoral model, with a center of rotation being as a function of at least one of a pelvic implant and a femoral implant.

10. The method according to claim 1, wherein aligning the first femoral model with the frame of reference comprises (1) rotating the first femoral model with respect to a center of rotation taken before initiation of hip joint replacement such that a mechanical axis of the first femoral model is parallel to a longitudinal axis of the frame of reference, and (2) rotating a femoral plane of the first femoral model with respect to the mechanical axis of the first femoral model such that the femoral plane of the first femoral model is parallel to a frontal plane of the frame of reference; and aligning the second femoral model with the frame of reference comprises (1) rotating the second femoral model with respect to a center of rotation taken after initiation of hip joint replacement such that a mechanical axis of the second femoral model is parallel to the longitudinal axis of the frame of reference, and (2) rotating a femoral plane of the second femoral model with respect to the mechanical axis of the second femoral model such that the femoral plane of the second femoral model is parallel to the frontal plane of the frame of reference.

11. The method according to claim 10, wherein digitizing the first femoral model comprises digitizing the mechanical axis, an anatomical axis, and the femoral plane in which both the mechanical axis and the anatomical axis lie.

12. The method according to claim 11, wherein digitizing the second femoral model comprises digitizing the mechanical axis, an anatomical axis, and the femoral plane in which both the mechanical axis and the anatomical axis lie, after initiation of hip joint replacement.

13. The method according to claim 1, wherein calculating surgical parameters comprises calculating a limb length discrepancy by projecting a mechanical axis of the first femoral model on a plane supporting the patient to form a longitudinal axis, and by measuring a distance on the longitudinal axis between a femoral landmark point taken before and after initiation of hip joint replacement.

14. The method according to claim 3, wherein calculating surgical parameters comprises calculating a medio-lateral offset by measuring a distance on the medio-lateral axis between a femoral landmark point taken before and after initiation of hip joint replacement.

15. A CAS system for measuring surgical parameters during hip replacement surgery to guide an operator in inserting a hip joint implant into a femur, comprising:
at least a first tracking reference in fixed relation with the pelvis, the first tracking reference being trackable to form a frame of reference;
a registration tool being trackable;
a sensor apparatus for tracking at least the first tracking reference and the registration tool; and
a controller unit for receiving tracking data for at least the first trackable reference and the registration tool, the controller unit having:
a position and orientation calculator for calculating from the tracking data a position and orientation of at least the pelvic tracking reference to track the frame of reference, and of the registration tool to produce femoral models at a first and a second sequential operative steps;
an alignment adjustor for receiving the femoral models as a function of at least the first tracking reference, and for aligning the femoral model of the second operative step and the femoral model of the first operative step with the frame of reference; and
a surgical parameter calculator for calculating surgical parameters as a function of the femoral model of the first operative step and the femoral model of the second operative step as aligned by the alignment adjustor.

16. The CAS system according to claim 15, wherein the position and orientation calculator produces the frame of reference from a normal of a plane supporting the patient in a given posture.

17. The CAS system according to claim 16, wherein the frame of reference comprises a medio-lateral axis being the normal of the plane when the patient is in lateral decubitus.

18. The CAS system according to claim 17, wherein the position and orientation calculator produces the frame of reference from a mechanical axis of the femur, with a projection of the mechanical axis on the plane and the normal of the plane forming two axes of the frame of reference.

19. The CAS system according to claim 15, wherein the first femoral model comprises an anatomical axis for the femur passing through a point on the greater trochanter and any one of a point on the patella and a midpoint of the condyles.

20. The CAS system according to claim 15, wherein the first femoral model comprises a mechanical axis passing through a center of rotation of the femur and any one of a point on the patella and a midpoint of the condyles.
